(19) United States
(12) Patent Application Publication
(10) Pub. No.: US 2026/0225090 A1
(43) Pub. Date: Aug. 6, 2026

(54) SELECTIVE HYDROGENATION

(71) Applicant: DSM IP ASSETS B.V., Maastricht (NL)

(72) Inventors: Werner BONRATH, Kaiseraugst (CH); Hans-Joachim DREXLER, Rostock (DE); Detlef HELLER, Rostock (DE); Nora JANNSEN, Rostock (DE); Jonathan Alan MEDLOCK, Kaiseraugst (CH); Marc-André MUELLER, Kaiseraugst (CH); Cornelia PRIBBENOW, Rostock (DE); Carmen SELLE, Rostock (DE)

(21) Appl. No.: 19/150,685

(22) PCT Filed: Feb. 2, 2024
(86) PCT No.: PCT/EP2024/052580

§ 371 Date: Jul. 24, 2025

(30) Foreign Application Priority Data
Feb. 2, 2023 (EP) 23154598.9

(51) Int. Cl. B01J 31/24

(52) U.S. Cl. CPC B01J 31/2409; B01J 2231/645; B01J 2531/842

(57) ABSTRACT

The present invention relates to the use of a specific homogenous catalyst for the partial (selective) hydrogenation of a carbon-carbon triple bond.

The present invention relates to the use of a specific homogenous catalyst for the partial (selective) hydrogenation of a carbon-carbon triple bond.

The catalyst is used for the selective hydrogenation, i.e. for the hydrogenation of alkynes to alkenes. Thus, if a compound contains a double bond as well as a triple bond, only the triple bond is reduced to a double bond.

Homogeneous catalysis refers to reactions where the catalyst is in the same phase as the reactants, principally in solution.

The aim of the present work was to improve the selectivity of selective hydrogenation reactions catalyzed by such catalysts.

It was found out that the transition metal catalyst with a specific bidentate phosphine ligand system shows a good efficiency of the hydrogenation while using mild reaction conditions.

The catalyst used in the selective hydrogenation according to the present invention has the following formula (I)

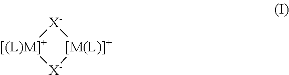

wherein
M is Rh or Ir, and
L is a bidentate phosphine ligand of formula (II)

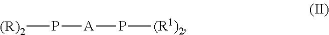

wherein
R is a substituted or unsubstituted cyclohexyl, substituted or unsubstituted phenyl, substituted or unsubstituted naphthyl, substituted or unsubstituted anthracenyl, anisyl, tert-butyl or isopropyl, and
R¹ is a substituted or unsubstituted cyclohexyl, substituted or unsubstituted phenyl, substituted or unsubstituted naphthyl, substituted or unsubstituted anthracenyl, anisyl, tert-butyl or isopropyl, and
A is a bridging moiety chosen from the group consisting of
—(CH₂)ₘ—, wherein m is an integer of value 1-6;

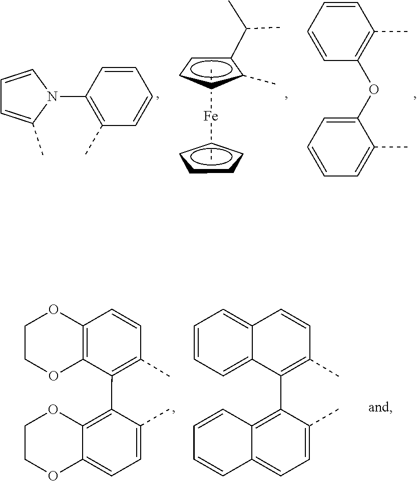

X⁻ is a halide, ⁻OAc, ⁻OH or ⁻OCH₃
and wherein any dotted line in formulae represents the bond by which the substituent is bound to the rest of the molecule.

The catalyst of the present invention is used in selective catalytic hydrogenation of starting material, especially of starting material comprising a carbon-carbon triple bond, more especially of alkynol compounds, especially preferred α-alkynol compounds.

Therefore, the present invention relates in a first aspect to a selective hydrogenation (H) using at least one catalyst of formula (I)

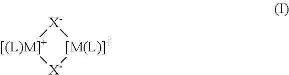

wherein
M is Rh or Ir, and
L is a bidentate phosphine ligand of formula (II)

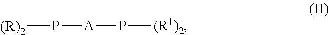

wherein
R is a substituted or unsubstituted cyclohexyl, substituted or unsubstituted phenyl, substituted or unsubstituted naphthyl, substituted or unsubstituted anthracenyl, anisyl, tert-butyl or isopropyl, and
R¹ is a substituted or unsubstituted cyclohexyl, substituted or unsubstituted phenyl, substituted or unsubstituted naphthyl, substituted or unsubstituted anthracenyl, anisyl, tert-butyl or isopropyl, and
A is a bridging moiety chosen from the group consisting of
—(CH₂)ₘ— wherein m is an integer of value 1-6;

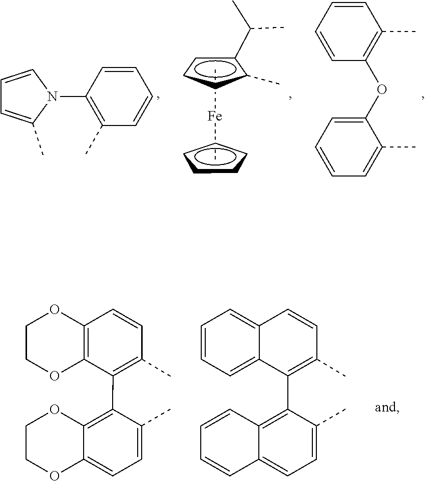

X⁻ is a halide, ⁻OAc, ⁻OH or ⁻OCH₃
and wherein any dotted line in formulae represents the bond by which the substituent is bound to the rest of the molecule.

Therefore, the present invention also relates to a selective hydrogenation (H1), which the selective hydrogenation (H), wherein starting material comprising a carbon-carbon triple bond are hydrogenated selectively.

Therefore, the present invention also relates to a selective hydrogenation (H2), which the selective hydrogenation (H) or (H1), wherein alkynol compounds are hydrogenated selectively.

Therefore, the present invention also relates to a selective hydrogenation (H2′), which the selective hydrogenation (H) or (H1), wherein α-alkynol compounds are hydrogenated selectively.

Preferably, the present invention also relates to a process of selective hydrogenation of a compound of formula (III)

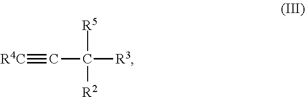

wherein
R² is a linear or branched C₁-C₃₅-alkyl; or linear or branched C₂-C₃₅-alkenyl moiety, wherein the C chain can be substituted, and
R³ is H or a linear or branched C₁-C₄-alkyl, wherein the C chain can be substituted, and
R⁴ is H or a cyclic, linear or branched C₁-C₆-alkyl, wherein the C chain can be substituted; or a C₅-C₁₂-cyclic aromatic moiety, which can be substituted, and
R⁵ is H or OH or a OC₁-C₄-alkyl; or a O(CO)C₁-C₄-alkyl.

The product of the selective hydrogenation is the compound of formula (IV)

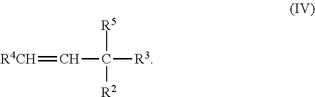

wherein
R², R³, R⁴ and R⁵ have the same meaning as defined in formula (III).

More preferably, the present invention also relates to a process of selective hydrogenation of a compound of formula (III)

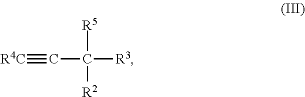

wherein
R² is a linear or branched C₁-C₃₀-alkyl; linear or branched C₂-C₃₀-alkenyl moiety, wherein the C chain can be substituted, and
R³ is a C₁-C₂-alkyl, and
R⁴ is H or a cyclic, linear or branched C₁-C₅-alkyl, wherein the C chain can be substituted, and
R⁵ is OH or a O(CO)C₁-C₂-alkyl
is hydrogenated selectively to a compound of the formula (IV)

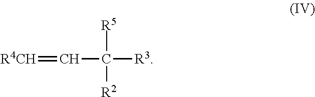

Even more preferably, the present invention also relates to a process of selective hydrogenation of a compound of formula (III)

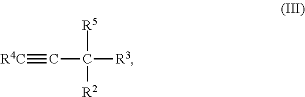

wherein
R² is a linear or branched C₁-C₂₀-alkyl; or linear or branched C₂-C₂₀-alkenyl moiety, wherein the C chain can be substituted, and
R³ is a C₁-C₂-alkyl, and
R⁴ is H, and
R⁵ is OH or a O(CO)C₁-C₂-alkyl
is hydrogenated selectively to a compound of the formula (IV).

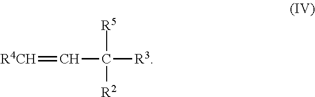

Most preferred compounds of formula (III) are the following of formula (IIIa) to (IIId)

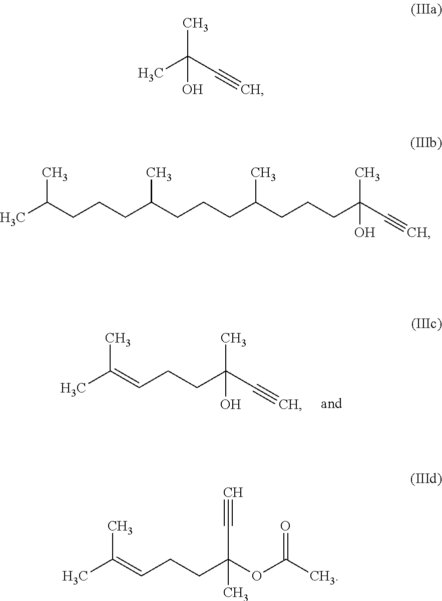

Therefore, the present invention also relates to a selective hydrogenation (H3), which is the hydrogenation (H), (H1) or (H2), wherein a compound of formula (III)

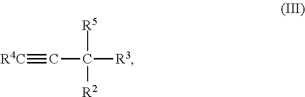

wherein
R² is a linear or branched C₁-C₃₅-alkyl; or linear or branched C₂-C₃₅-alkenyl moiety, wherein the C chain can be substituted, and
R³ is H or a linear or branched C₁-C₄-alkyl, wherein the C chain can be substituted, and
R⁴ is H or a cyclic, linear or branched C₁-C₆-alkyl, wherein the C chain can be substituted; or a C₅-C₁₂-cyclic aromatic moiety, which can be substituted, and
R⁵ is a H or OH or a OC₁-C₄-alkyl or a O(CO)C₁-C₄-alkyl,
is hydrogenated selectively.

Therefore, the present invention also relates to a selective hydrogenation (H3′), which is the hydrogenation (H), (H1), (H2) or (H2′), wherein a compound of formula (III)

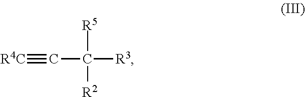

wherein
R² is a linear or branched C₁-C₃₀-alkyl; or linear or branched C₂-C₃₀-alkenyl moiety, wherein the C chain can be substituted, and
R³ is a C₁-C₂-alkyl, and
R⁴ is H or a cyclic, linear or branched C₁-C₆-alkyl, wherein the C chain can be substituted, and
R⁵ is OH or a O(CO)C₁-C₂-alkyl,
is hydrogenated selectively.

Therefore, the present invention also relates to a selective hydrogenation (H3″), which is the hydrogenation (H), (H1), (H2) or (H2′), wherein a compound of formula (III)

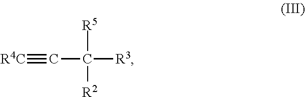

wherein
R² is a linear or branched C₁-C₂₀-alkyl; or linear or branched C₂-C₂₀-alkenyl moiety, wherein the C chain can be substituted, and
R³ is a C₁-C₂-alkyl, and
R⁴ is H, and
R⁵ is OH or a O(CO)C₁-C₂-alkyl,
is hydrogenated selectively.

Therefore, the present invention also relates to a selective hydrogenation (H3′″), which is the hydrogenation (H), (H1), (H2) or (H2′), wherein a compound of formula (IIIa), (IIIb), (IIIc) or (IIId)

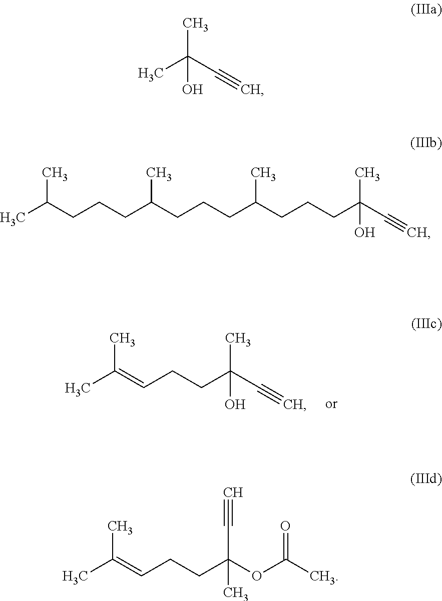

is hydrogenated selectively to a compound of the formula (IVa), (IVb), (IVc) or (IVd), respectively.

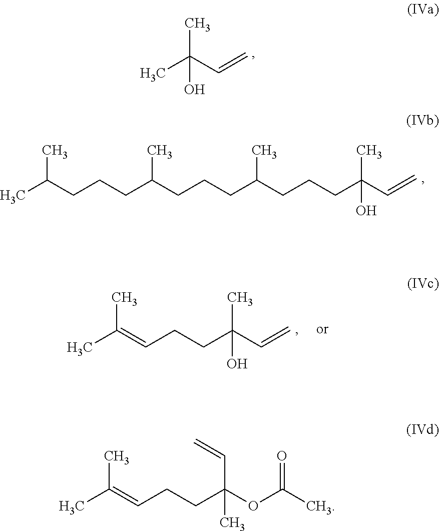

As stated above, the selective hydrogenation according to the present invention is carried out using a specific catalyst of formula (I).

Preferred catalysts of formula (I) are those, wherein M is Rh.

It is well known that the applied catalyst generally changes during the catalytic cycle, for example between monomeric, dimeric and oligomeric species. Therefore, the catalyst of formula (I) is that which is initially applied in the selective hydrogenation reaction.

Therefore, the present invention also relates to a selective hydrogenation (H4), which is the hydrogenation (H), (H1), (H2), (H2′), (H3), (H3′), (H3″) or (H3′″), wherein a catalyst of formula (I), wherein M is Rh is used.

Preferred catalysts of formula (I) are those, wherein L is a bidentate phosphine ligand chosen from the group consisting of the following ligands of formulae (IIa) to (III)

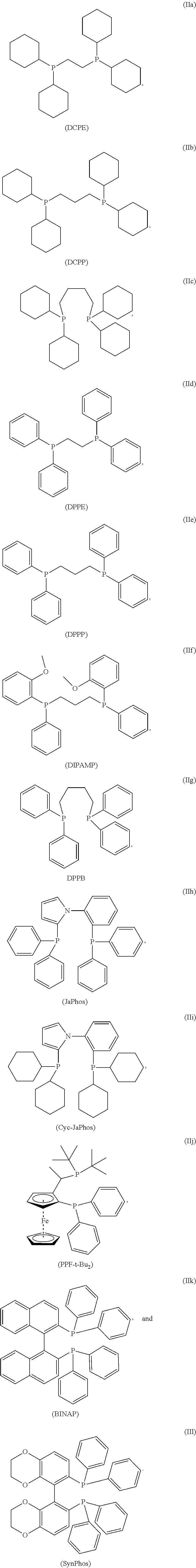

More preferred catalysts of formula (I) are those, wherein
L is a bidentate phosphine ligand chosen from the group consisting of the following ligands of formulae

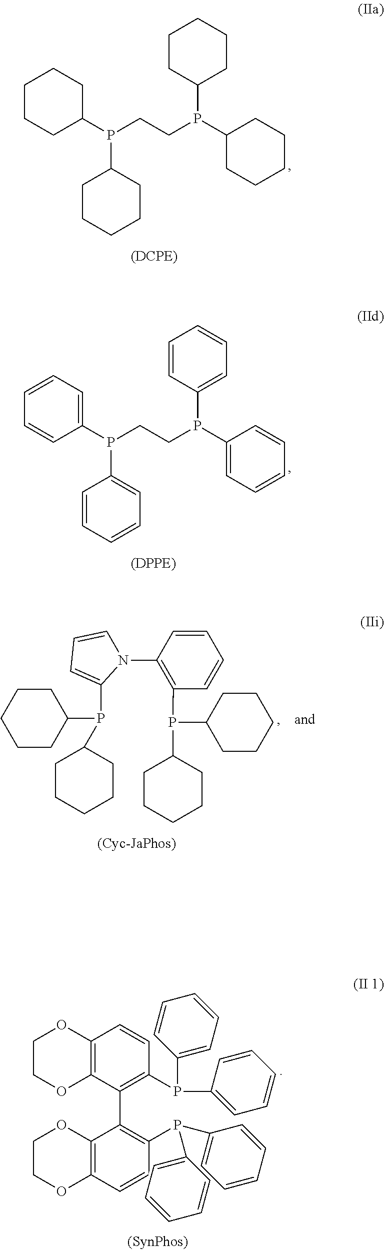 [SMILES: C1CCC(P(CCP(C2CCCCC2)C2CCCCC2)C2CCCCC2)CC1 ; c1ccc(P(CCP(c2ccccc2)c2ccccc2)c2ccccc2)cc1 ; c1ccc(P(C2CCCCC2)C2CCCCC2)c(-n2cccc2P(C2CCCCC2)C2CCCCC2)c1 ; c1ccc(P(c2ccccc2)c2ccc3c(c2-c2c(P(c4ccccc4)c4ccccc4)ccc4c2OCCO4)OCCO3)cc1]

Therefore, the present invention also relates to a selective hydrogenation (H5), which is the hydrogenation (H), (H1), (H2), (H2′), (H3), (H3′), (H3″), (H3′″) or (H4), wherein a catalyst of formula (I), wherein
L is a bidentate phosphine ligand chosen from the group consisting of the ligands of formulae (IIa) to (III)

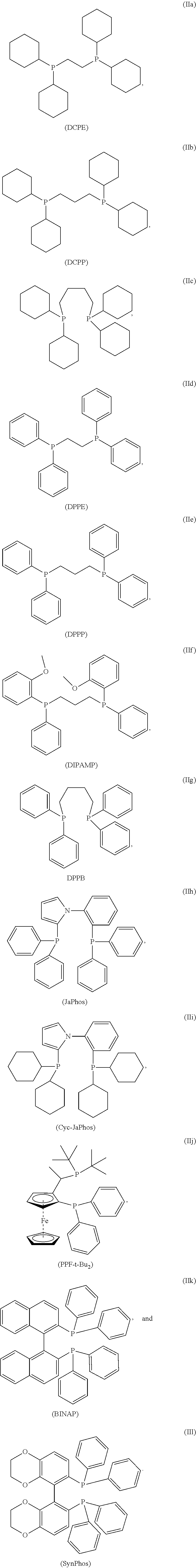

is used.

Therefore, the present invention also relates to a selective hydrogenation (H5′), which is the hydrogenation (H), (H1), (H2), (H2′), (H3), (H3′), (H3″), (H3′″) or (H4), wherein a catalyst of formula (I), wherein
L is a bidentate phosphine ligand chosen from the group consisting of the following ligands

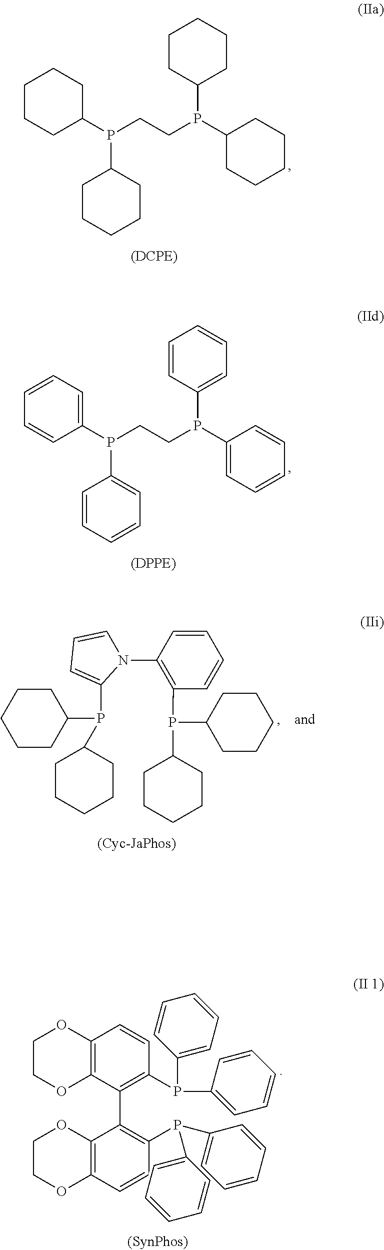 [SMILES: C1CCC(P(CCP(C2CCCCC2)C2CCCCC2)C2CCCCC2)CC1 ; c1ccc(P(CCP(c2ccccc2)c2ccccc2)c2ccccc2)cc1 ; c1ccc(P(C2CCCCC2)C2CCCCC2)c(-n2cccc2P(C2CCCCC2)C2CCCCC2)c1 ; c1ccc(P(c2ccccc2)c2ccc3c(c2-c2c(P(c4ccccc4)c4ccccc4)ccc4c2OCCO4)OCCO3)cc1]

is used.

Preferred catalysts are those, wherein X⁻ is Cl⁻, Br⁻, I⁻, ⁻OAc, ⁻OH or ⁻OCH₃.

is halide, ⁻OAc, ⁻OH or ⁻OCH₃.

More preferred catalysts are those, wherein X⁻ is Cl⁻ or Br⁻.

Most preferred catalyst are those, wherein X⁻ is Cl⁻.

Therefore, the present invention also relates to a selective hydrogenation (H6), which is the hydrogenation (H), (H1), (H2), (H2′), (H3), (H3′), (H3″), (H3′″), (H4), (H5) or (H5′), wherein X⁻ is chosen from the group consisting of Cl⁻, Br⁻, I⁻, ⁻OAc, ⁻OH and ⁻OCH₃.

Therefore, the present invention also relates to a selective hydrogenation (H6′), which is the hydrogenation (H), (H1), (H2), (H2′), (H3), (H3′), (H3″), (H3′″), (H4), (H5) or (H5′), wherein X⁻ is chosen from the group consisting of Cl⁻ and Br⁻.

Therefore, the present invention also relates to a selective hydrogenation (H6″), which is the hydrogenation (H), (H1), (H2), (H2′), (H3), (H3′), (H3″), (H3′″), (H4), (H5) or (H5′), wherein X⁻ is Cl⁻.

The catalysts, which are used in the hydrogenation according to the present invention are made as disclosed in the prior art.

The hydrogenation according to the present invention can be carried without any solvent.

Therefore, the present invention also relates to a selective hydrogenation (H7), which is the hydrogenation (H), (H1), (H2), (H2′), (H3), (H3′), (H3″), (H3′″), (H4), (H5), (H5′), (H6), (H6′) or (H6″), wherein the hydrogenation is carried out without any solvent.

The hydrogenation according to the present invention can be carried out in the presence of at least one inert solvent.

The hydrogenation can be carried out in a solvent (or mixture of solvents). Suitable solvents are alkanes, esters, carbonates, lactones, ethers, amides, hydrocarbons, halogenated hydrocarbons, water and alcohols. Preferred solvents are water, hexane, CH₂Cl₂, toluene, ethyl acetate, THF (=tetrahydrofuran), 2-Me-THF (=2-methyl-tetrahydrofuran), cyclopentyl methyl ether (=CPME), methanol, ethanol and isopropanol, especially preferred solvents are methanol, water and hexane.

Therefore, the present invention also relates to a selective hydrogenation (H8), which is the hydrogenation (H), (H1), (H2), (H2′), (H3), (H3′), (H3″), (H3′″), (H4), (H5), (H5′), (H6), (H6′) or (H6″), wherein the hydrogenation is carried out in at least one solvent.

Therefore, the present invention also relates to a selective hydrogenation (H8′), which is the hydrogenation (H8), wherein the solvent is chosen from the group consisting of alkanes, esters, carbonates, lactones, ethers, amides, hydrocarbons, halogenated hydrocarbons, water and alcohols.

Therefore, the present invention also relates to a selective hydrogenation (H8″), which is the hydrogenation (H8), wherein the solvent is chosen from the group consisting of water, hexane, CH₂Cl₂, toluene, ethyl acetate, THF, 2-Me-THF, cyclopentyl methyl ether, methanol, ethanol and isopropanol.

Therefore, the present invention also relates to a selective hydrogenation (H8′″), which is the hydrogenation (H8), wherein the solvent is chosen from the group consisting of methanol, water and hexane.

The catalyst of formula (I) according to the present invention is usually used in an amount of 0.001-1 mol-% (preferably 0.001-0.5 mol-%) (based on moles of the compounds of formula (III)).

Therefore, the present invention also relates to a selective hydrogenation (H9), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″) or (H8′″), wherein the at least one catalyst of formula (I) is used in an amount of 0.001-1 mol-% (based on moles of the compounds of formula (III)).

Therefore, the present invention also relates to a selective hydrogenation (H9′), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″) or (H8′″), wherein the at least one catalyst of formula (I) is used in an amount of 0.001-0.5 mol-% (based on moles of the compounds of formula (III)).

The hydrogenation process can be carried out with (pure) H₂ gas or with a gas, which comprises H₂. Preferably, the hydrogenation process according to the present invention is carried out with (pure) H₂ gas.

Therefore, the present invention also relates to a selective hydrogenation (H10), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″), (H8′″), (H9) or (H9′), wherein the hydrogenation is carried out with (pure) H₂ gas or with a gas, which comprises H₂.

Therefore, the present invention also relates to a selective hydrogenation (H10′), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″), (H8′″), (H9) or (H9′), wherein the hydrogenation is carried out with H₂ gas.

The hydrogenation process can be carried out at ambient pressure as well as at elevated pressure. Preferably, the hydrogenation process according to the present invention is carried out at a pressure of 1-50 bar, more preferably at 1-30 bar. Usually, the reaction is carried out in an autoclave (or any other vessel, which can resist the pressure).

Therefore, the present invention also relates to a selective hydrogenation (H11), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″), (H8′″), (H9), (H9′), (H10) or (H10′), wherein the hydrogenation is carried out at ambient pressure.

Therefore the present invention also relates to a selective hydrogenation (H11′), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″), (H8′″), (H9), (H9′), (H10) or (H10′), wherein the hydrogenation is carried out at a pressure of 1-50 bar, preferably at 1-30 bar.

The hydrogenation is usually carried out at a temperature of −10-150° C. (preferably 10 100° C.)

Therefore the present invention also relates to a selective hydrogenation (H12), which is the hydrogenation (H), (H1), (H2), (H2′), (H3), (H3′), (H3″), (H3′″), (H4), (H5), (H5′), (H6), (H6′), (H6″), (H7), (H8), (H8′), (H8″), (H8′″), (H9), (H9′), (H10), (H10′), (H11) or (H11′), wherein the hydrogenation is carried out at a temperature of −10-150° C.

The following examples serve to illustrate the invention. The temperature is given in ° C. and all percentages are related to the weight.

### EXAMPLES

### General Catalyst Synthesis (Example 1) Synthesis of[Rh(DPPE)(μ2-Cl)]₂

A Schlenk tube was charged with [Rh(cod)(μ₂-Cl)]₂ (185.6 mg, 0.376 mmol)(cod=1,5-cyclooctadiene), dissolved in 5 ml of toluene and heated to 70° C. DPPE (300.0 mg, 0.753 mmol) dissolved in toluene (5 mL) was added dropwise within 2 h. The reaction mixture was stirred for another 3 h at 125° C. Subsequently the solvent was evaporated and the precipitation dried under vacuum and the catalyst ([Rh(DPPE)(μ₂-Cl)]₂) was obtained.

¹H NMR (300 MHz, THF-d₈, 297 K): δ=1.98 (dd, J=19.21 Hz, J=1.02 Hz, 8H), 7.13-7.26 (m, 24H), 7.87-7.93 (m, 16H) ppm.

³¹P NMR (121 MHz, THF-d₈, 297 K): δ=72.9 (d, J_{P-Rh}=198.3 Hz) ppm.

All other used catalyst in the following examples have been produced in analogy to the process of example 1.

### HYDROGENATION EXAMPLES

### Example 2

0.5 mmol Dehydroisophytol (compound of formula (IIIb)), 7.5 ml methanol and 0.005 mmol of the [Rh(DPPE)(μ₂-Cl)]₂, which was produced as disclosed in example 1, were put into an autoclave. The mixture was stirred and the H₂ was added in form of H₂ gas.

The autoclave was thermostated to 25°.C and hydrogen pressure released after 13 min. Isophytol was obtained in a yield of 94.2%.

### Example 3

440 mmol Dehydroisophytol (compound of formula (IIIb)), 650 ml methanol and 0.044 mmol of the [Rh(DPPE)(μ2-Cl)]₂, which was produced as disclosed in example 1, were put into an autoclave. The mixture was stirred and the H₂ was added in form of H₂ gas till a pressure of 3.5 bar.

The autoclave was thermostated to 25° C. and hydrogen pressure released after 102 min. Isophytol was obtained in a yield of 91.4%.

The following examples have been made in analogy to the process of example 2 (the tables 1 to 4 list the difference to the reaction conditions)

TABLE 1

| | | t | DIP* | IP* | DiIP* |
|---|---|---|---|---|---|
| Exp. | Cat. | [min] | [%] | [%] | [%] |
| 4 | [Rh(cyc-Japhos)(μ₂-Cl)]₂ | 8 | 0.9 | 81.3 | 17.8 |
| 5 | [Rh(cyc-Japhos)(μ₂-Cl)]₂ | 15 | 0 | 81.1 | 18.9 |
| 6 | [Rh(SynPhos)(μ₂-Cl)]₂ | 260 | 9.6 | 85.7 | 4.7 |

Table 1.
*DIP = Dehydroisophytol; IP = isophytol; DiIP = perhydro-genated dehydroisophytol; =3,7,11,15-tetramethyl-hexadecan-3-ol.

TABLE 2
Table 2.

| | | | t | DIP* | IP* | DiIP* |
|---|---|---|---|---|---|---|
| No | Cat. | Solvent | [min] | [%] | [%] | [%] |
| 7 | [Rh(DCPE)(μ₂-Cl)]₂ | n-hexane | 1260 | 0 | 88.0 | 12.0 |

*DIP = Dehydroisophytol; IP = isophytol; DiIP = perhydrogenated dehydroisophytol; =3,7,11,15-tetramethylhexadecan-3-ol.

TABLE 3
Table 3.

| | | T | t | DIP* | IP* | DiIP* |
|---|---|---|---|---|---|---|
| No | Cat. | [° C.] | [min] | [%] | [%] | [%] |
| 8 | [Rh(DCPE)(μ₂-Cl)]₂ | 5 | 19 | 21.5 | 77.4 | 1.1 |
| 9 | [Rh(DCPE)(μ₂-Cl)]₂ | 5 | 45 | 0 | 95.5 | 4.4 |
| 10 | [Rh(DCPE)(μ₂-Cl)]₂ | 45 | 7.5 | 6.1 | 91.0 | 2.9 |

*DIP = Dehydroisophytol; IP = isophytol; DiIP = perhydrogenated dehydroisophytol; =3,7,11,15-tetramethylhexadecan-3-ol.

TABLE 4
Table 4.

| | | | H₂ | t | DIP* | IP* | DiIP* |
|---|---|---|---|---|---|---|---|
| No | Cat. | S:C | [bar] | [min] | [%] | [%] | [%] |
| 11 | [Rh(DCPE)(μ₂-Cl)]₂ | 800 | 1 | 90 | 1.7 | 94.5 | 3.8 |
| 12 | [Rh(DCPE)(μ₂-Cl)]₂ | 3000 | 1 | 660 | 0.3 | 92.3 | 7.3 |

*DIP = Dehydroisophytol; IP = isophytol; DiIP = perhydrogenated dehydroisophytol; =3,7,11,15-tetramethylhexadecan-3-ol.

1. Selective hydrogenation using at least one catalyst of formula (I) 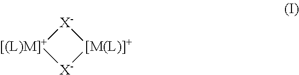
wherein
M is Rh or Ir, and
L is a bidentate phosphine ligand of formula (II) 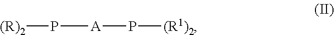
wherein
R is a substituted or unsubstituted cyclohexyl, substituted or unsubstituted phenyl, substituted or unsubstituted naphthyl, substituted or unsubstituted anthracenyl, anisyl, tert-butyl or isopropyl, and
R¹ is a substituted or unsubstituted cyclohexyl, substituted or unsubstituted phenyl, substituted or unsubstituted naphthyl, substituted or unsubstituted anthracenyl, anisyl, tert-butyl or isopropyl, and
A is a bridging moiety chosen from the group consisting of
—(CH₂)ₘ—, wherein m is an integer of value 1-6; 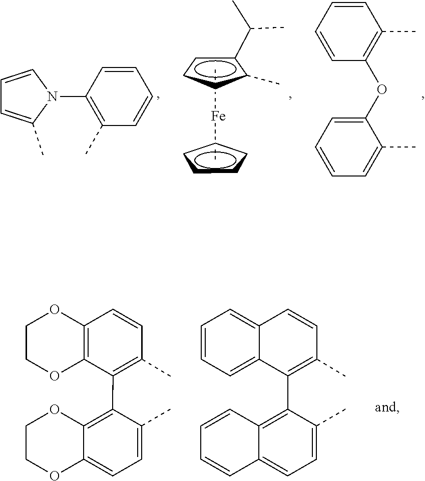
X⁻ is a halide, ⁻OAc, ⁻OH or ⁻OCH₃
and wherein any dotted line in formulae represents the bond by which the substituent is bound to the rest of the molecule.

2. Selective hydrogenation according to claim 1, wherein a starting material comprising a carbon-carbon triple bond is hydrogenated selectively.

3. Selective hydrogenation according to claim 1, wherein a compound of formula (III) 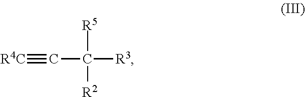
wherein
R² is a linear or branched C₁-C₃₅-alkyl; or linear or branched C₂-C₃₅-alkenyl moiety, wherein the C chain can be substituted, and
R³ is H or a linear or branched C₁-C₄-alkyl, wherein the C chain can be substituted, and
R⁴ is H or a cyclic, linear or branched C₁-C₆-alkyl, wherein the C chain can be substituted; or a C₅-C₁₂-cyclic aromatic moiety, which can be substituted, and
R⁵ is H or OH or a OC₁-C₄-alkyl; or a O(CO)C₁-C₄-alkyl,
is hydrogenated selectively to a compound of the formula (IV)

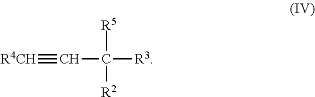

4. Selective hydrogenation according to claim 1, wherein a compound of formula (III) 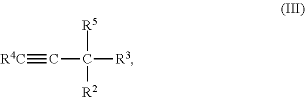
wherein
R² is a linear or branched C₁-C₂₀-alkyl; or linear or branched C₂-C₂₀-alkenyl moiety, wherein the C chain can be substituted, and
R³ is a C₁-C₂-alkyl, and
R⁴ is H, and
R⁵ is OH or a O(CO)C₁-C₂alkyl,
is hydrogenated selectively to a compound of the formula (IV)

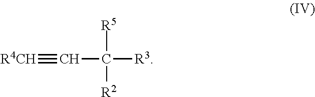

5. Selective hydrogenation according to claim 1, wherein a catalyst of formula (I), wherein M is Rh is used.

6. Selective hydrogenation according to claim 1, wherein a catalyst of formula (I), wherein
L is a bidentate phosphine ligand chosen from the group consisting of the ligands of formula (IIa) to (III) 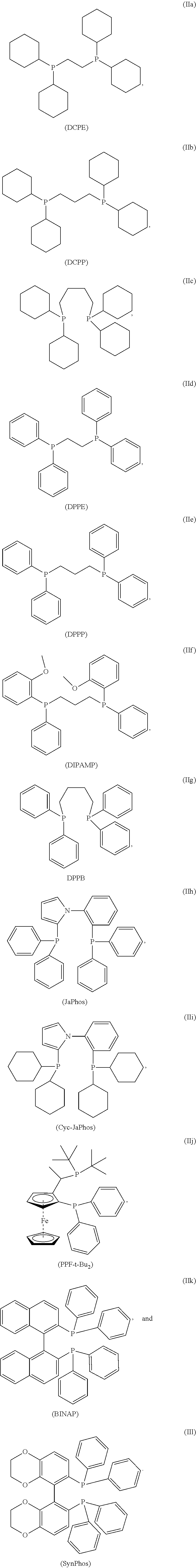
is used.

7. Selective hydrogenation according to claim 1, wherein a catalyst of formula (I), wherein
L is a bidentate phosphine ligand chosen from the group consisting of the ligands of formula 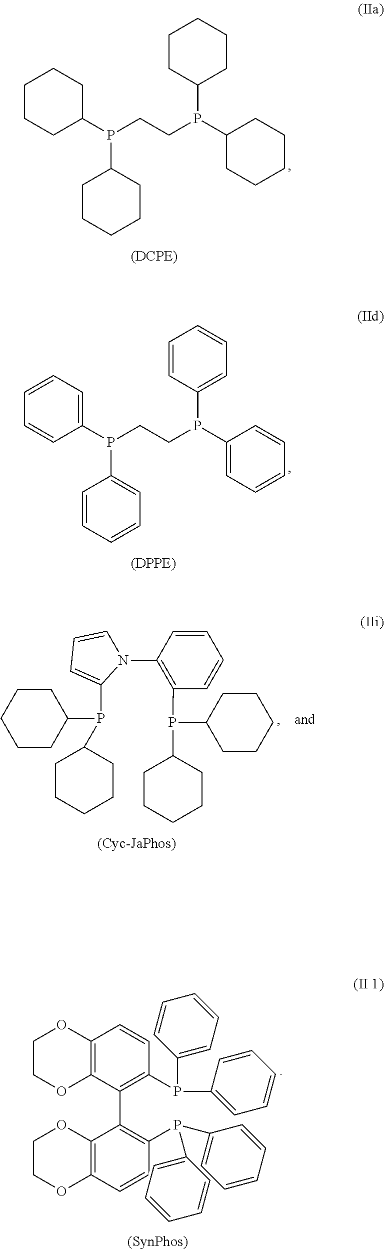 [SMILES: C1CCC(P(CCP(C2CCCCC2)C2CCCCC2)C2CCCCC2)CC1 ; c1ccc(P(CCP(c2ccccc2)c2ccccc2)c2ccccc2)cc1 ; c1ccc(P(C2CCCCC2)C2CCCCC2)c(-n2cccc2P(C2CCCCC2)C2CCCCC2)c1 ; c1ccc(P(c2ccccc2)c2ccc3c(c2-c2c(P(c4ccccc4)c4ccccc4)ccc4c2OCCO4)OCCO3)cc1]
is used.

8. Selective hydrogenation according to claim 1, wherein a catalyst of formula (I), wherein X⁻ is chosen from the group consisting of Cl⁻, Br⁻, I⁻, ⁻OAc, ⁻OH and or ⁻OCH₃.

9. Selective hydrogenation according to claim 1, wherein a catalyst of formula (I), wherein X⁻ is chosen from the group consisting of Cl⁻ and Br⁻.

10. Selective hydrogenation according to claim 1, wherein the hydrogenation is carried out without any solvent.

11. Selective hydrogenation according to claim 1, wherein the hydrogenation is carried out in at least one solvent.

12. Selective hydrogenation according to claim 1, wherein the at least one catalyst of formula (I) is used in an amount of 0.001-1 mol-% (based on the moles of the compounds of formula (III)).

13. Selective hydrogenation according to claim 1, wherein the hydrogenation is carried out with (pure) H₂ gas or with a gas, which comprises H₂.

14. Selective hydrogenation according to claim 1, wherein the hydrogenation is carried out at a pressure of 1-50 bar.

15. Selective hydrogenation according to claim 1, wherein the hydrogenation is carried out carried at a temperature of −10-150° C.
